(19) United States
(12) Patent Application Publication
(10) Pub. No.: US 2006/0224153 A1
(43) Pub. Date: Oct. 5, 2006

(54) Catheter system for the treatment of atrial fibrillation

(76) Inventors: Robert E. Fischell, Dayton, MD (US); Tim A. Fischell, Kalamazoo, MI (US)

Correspondence Address: Robert E. Fischell, 14600 Viburnum Dr, Dayton, MD, 21036, US

(21) Appl. No.: 11/098,636

(22) Filed: Apr. 5, 2005

(51) Int. Cl. A61B 18/14

(52) U.S. Cl. 606/41

(57) ABSTRACT

Disclosed are a means and method for rapidly and accurately positioning a toroidal balloon with its distal surface placed tightly against the endocardial surface of the left atrium at a location that is close to the ostium of a pulmonary vein. On the exterior surface of the toroidal balloon can be an electrically conducting wire that is capable of causing rf energy to be placed into the tissue of the left atrium so as to ablate that tissue to alter the conduction of aberrant electrical signals of the heart that are associated with atrial fibrillation. The toroidal balloon is wrapped circumferentially around a tapered balloon that is placed into the pulmonary vein. This system can be applied successively to at least one or as many as all four of the pulmonary veins that enter the left atrium to treat the patient's atrial fibrillation.

### FIELD OF USE

This invention is in the field of methods and devices for the treatment of atrial fibrillation.

### BACKGROUND OF THE INVENTION

Atrial fibrillation is a disorder of the heart that (in the year 2005) affects more than 2 million Americans and a comparable number of patients outside the USA. There have been many treatments for this disorder including surgery and percutaneous catheter treatments that require mapping of the electrical signals within the heart and then ablative destruction of the region of the pulmonary vein that is in close proximity to the left atrium. The mapping procedure is often very difficult and time consuming, thus significantly extending the time required for the procedure. The surgical operation requires considerable skill and a great deal of time for that procedure. Furthermore, the recovery time from the surgical treatment is significantly greater than the recovery time from a percutaneous catheter procedure for the treatment of this disorder.

One of the significant problems in a catheter treatment for atrial fibrillation is accurately positioning the distal portion of the catheter to ablate the tissue required to treat the atrial fibrillation. Another problem when radio-frequency (rf) ablation is used is the creation of blood clots within the left atrium that can embolize to an artery in the brain thereby causing a stroke. Another serious limitation with a number of existing catheter systems for the treatment of atrial fibrillation is that pulmonary vein stenosis may occur over time with serious clinical consequences. What is really needed is a catheter-based treatment system that can be efficiently used to ablate the appropriate atrial tissue, and also minimizes the procedural risks of stroke and pulmonary vein stenosis.

### SUMMARY OF THE INVENTION

The present invention is designed to overcome the shortcomings of prior art methods and devices for the treatment of atrial fibrillation. Explicitly, the present invention is a catheter means and a method for rapidly and accurately positioning a toroidal balloon with its distal surface placed tightly against the endocardial surface of the left atrium at a location that is close to the ostium of a pulmonary vein. On the exterior surface of the toroidal balloon can be an electrically conducting wire that is capable of causing rf energy to be placed into the tissue of the left atrium so as to ablate that tissue to alter the conduction of aberrant electrical signals of the heart that are associated with atrial fibrillation. The toroidal balloon is actually wrapped circumferentially around a tapered balloon whose proximal diameter is just slightly less than or equal to the diameter near the ostium of the pulmonary vein into which the tapered balloon is inserted. The positions of the tapered balloon and its surrounding toroidal balloon are such that the ablation of the tissue of the left atrium occurs at a diameter that is approximately 1-4 mm greater than the diameter of the ostium of the pulmonary vein into which the tapered balloon is inserted. In this manner, only the tissue of the left atrium, surrounding the ostium of the pulmonary vein is ablated. Thus, injury to the pulmonary vein can be minimized in order to reduce the risk of iatrogenic pulmonary vein stenosis. This system can be applied successively to at least one or as many as all four of the pulmonary veins that enter the left atrium. The same type of catheter system can be used to ablate the endocardial surface of the right atrium centered around the ostium of the superior vena cava.

The method for accomplishing this catheter treatment to cure atrial fibrillation is generally as follows:
1) an introducer sheath is placed into the left or right femoral vein,
2) a guide wire is advanced through the femoral vein, through the inferior vena cava and then into the right atrium,
3) using standard trans-septal puncture technique, a guide wire is placed across the interatrial septum from the right into the left atrium,
4) further dilatation of the interatrial septum puncture site can be performed with a balloon angioplasty catheter, if necessary,
5) appropriate anticoagulation is given to minimize the risk of left atrial thrombus during the rest of the manipulations,
6) a special purpose guiding catheter including a dilator is advanced over the guide wire and through the inferior vena cava and into the right atrium,
7) the dilator and guiding catheter are then pushed through the interatrial septum from the right atrium into the left atrium, using the guide wire already placed in the left atrium,
8) the dilator is removed,
9) a specially designed two-balloon catheter is then advanced over the guide wire and through the guiding catheter until its distal end is situated in the left atrium,
10) the guide wire and/or the guiding catheter are manipulated until the guide wire is placed into the first targeted pulmonary vein,
11) the guidance of the guide wire and the balloon ablation catheter into each of the targeted pulmonary veins (up to four) can be guided by intra-cardiac or trans-esophageal ultrasound imaging, or other real-time imaging modalities,
12) the tapered balloon and the toroidal balloon are then advanced until the proximal portion of the tapered balloon is situated just proximal to the ostium of that pulmonary vein,
13) the tapered balloon and the toroidal balloon are then inflated to a pressure such that the proximal portion of the tapered balloon just fits within the ostial region of the pulmonary vein,
14) the balloons are then pushed forward until the distal surface of the toroidal balloon is in contact with, and applies some pressure onto, the endocardial surface of the left atrium surrounding the ostium of the pulmonary vein,
15) ablation of the tissue of the left atrium surrounding the ostium of the pulmonary vein is then applied by any well known method such as the use of rf energy, cryogenic cooling or heating with a high temperature fluid or heated electrical wire,
16) the application of the ablative means is then discontinued and the balloons are deflated,
17) if it is desired to ablate the region of the left atrium near the ostium of a second, third or fourth pulmonary vein, then the steps 10-16 above are repeated for each region where it is desired to perform tissue ablation of the left atrium,
18) if desired, the balloons are deflated, the catheter is pulled back into the right atrium and the region around the ostium of the superior vena cava is ablated in a manner as described in steps 10-16 above, and
19) when treatment of the last region of the left atrium (or superior vena cava) that is to be ablated is completed, all the apparatus used (viz., the guide wire, two-balloon catheter, guiding catheter and introducer sheath, etc.) are then removed from the patient's body.

It is believed that mapping of the electrical voltages within the heart may be accomplished before and/or after the ablative catheter procedure described herein. If that were done, then only those regions of the left atrium that are involved in the aberrant electrical signal causing the atrial fibrillation would be ablated. Unfortunately, mapping takes a great deal of procedure time and does not always provide a clear answer as to the region of the heart that must be ablated in order to stop the episodes of atrial fibrillation being experienced by the patient. It may be more advantageous merely to empirically treat the regions of the left atrium surrounding each of the four pulmonary veins that feed into the left atrium without the use of mapping. As mentioned above, it may also be desirable to treat the endocardial region of the right atrium in the vicinity of the ostium of the superior vena cava using the same catheter system. In all cases, the diameter of the tapered balloon can be adjusted at some time during the procedure so that it is approximately the diameter of the pulmonary vein near its ostium.

Visualization of the left and right atria and pulmonary veins is best accomplished by intracardial or esophageal ultrasound as is presently done in the treatment of atrial fibrillation. Fluoroscopy may also be utilized for improving the visualization of the left atrium and pulmonary veins. The optimal guiding catheter and/or guide wire to be used for such a procedure would be of the steerable type that is now well known in the field of interventional cardiology. By utilizing steerable equipment, placement of the guide wire and balloon catheter into each pulmonary vein could be accomplished more expeditiously. The guide wire used would typically have a diameter of 0.035 or 0.038 inches. The guiding catheter used for the procedure would typically have a diameter between 8 FR and 14 FR.

Thus one object of the present invention is to create a lesion of the tissue of the left atrium that is in close proximity to the ostium of at least one pulmonary vein thereby preventing the occurrence of atrial fibrillation, the procedure being performed by means of a percutaneously inserted catheter system.

Another object of this invention is to successively ablate atrial tissue in the left atrium that is in close proximity to more than one of the ostia of the pulmonary veins.

Still another object of this invention is to ablate the tissue of the left atrium around the ostia of the pulmonary veins by means of rf or resistive wire heating, cryogenic cooling, intense, localized ultrasonic vibration or by any other means that can create a lesion that will prevent the occurrence of atrial fibrillation.

Still another object of this invention is to ablate the tissue of the right atrium around the ostium of the superior vena cava.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a cross section of the right and left atria of a human heart showing a guiding catheter placed through the interatrial septum.

FIG. 2 illustrates a two-balloon catheter advanced over a guide wire into one of the pulmonary veins that enter the left atrium with the balloons being inflated.

FIG. 3 is a longitudinal cross section of a distal portion of the two-balloon catheter used for ablating a region of the left atrium that is in close proximity to the ostium of a pulmonary vein with the two balloons being inflated.

FIG. 4 is the cross section of the two-balloon catheter at section 4-4 of FIG. 3.

### DETAILED DESCRIPTION OF THE INVENTION

FIG. 1 is a cross section of the right and left atria of a human heart showing two of the four pulmonary veins that enter the left atrium. FIG. 1 does not attempt to accurately display the exact location of these two pulmonary veins. It should be understood that there are actually four pulmonary veins that enter the left atrium. FIG. 1 also shows a distal portion of a guiding catheter 10 that is designed explicitly for introduction through the left or right femoral vein; from there through the inferior vena cava, and from there into the right atrium. After the trans-septal placement of a guide wire 12 into the left atrium, the dilator 11 and the distal end of the guiding catheter 10 can be advanced over the guide wire 12 and through the interatrial septum into the left atrium. The dilator 11 can include a radiopaque metal marker ring (not shown) at or near its distal end to improve the visualization of the dilator 11 by means of fluoroscopy or echocardiography. After the distal end of the guiding catheter 10 has been placed into the left atrium, the dilator 11 is removed from the patient's body.

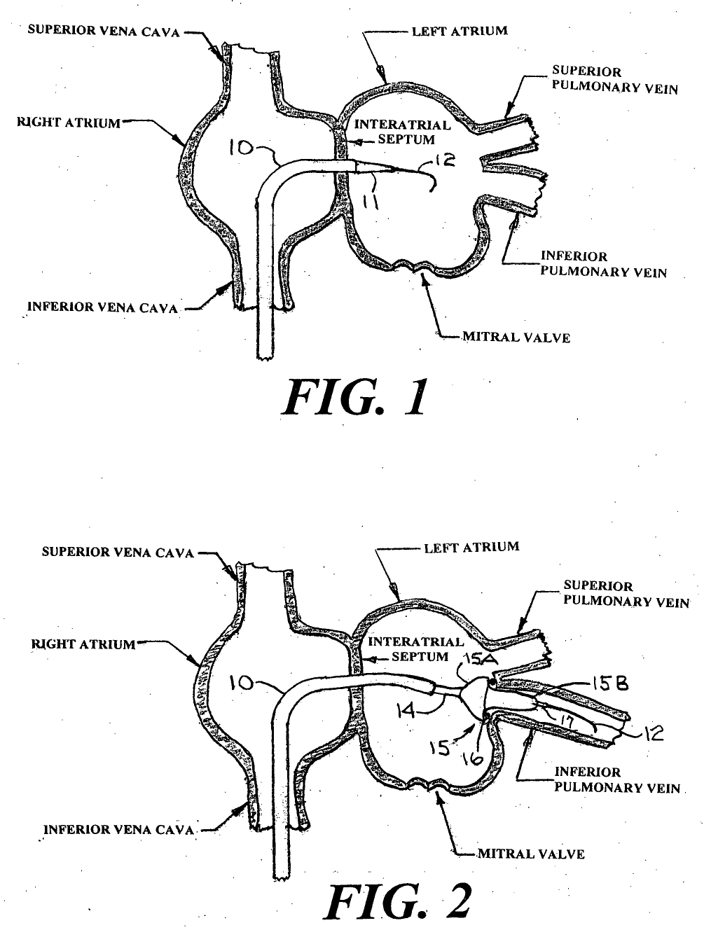

FIG. 2 shows the position of various parts of the two-balloon catheter system after the balloons 15A and 15 B have been inflated. The sequence of events before inflating the balloons 15 A and 15B begin with the removal of the dilator 11 from the patient's body. Then a pre-deployed, two-balloon catheter having a distal portion 15 is advanced through the guiding catheter 10 into the left atrium. After the guide wire 12 is advanced into a pulmonary vein, the two-balloon catheter's distal portion 15 is advanced over the guide wire 12 and into that pulmonary vein so that a proximal portion of the tapered balloon 15B is located just proximal to the ostium of the pulmonary vein. Both the tapered balloon 15B and the toroidal balloon 15A are then either separately or simultaneously inflated. The pressure applied to the somewhat compliant balloon material of each of the balloons 15A and 15B is such as to increase the diameter of the proximal portion of tapered balloon 15B so that its diameter is equal to or just slightly smaller than the diameter of the pulmonary vein just distal to its ostium. The proximal portion of the tapered balloon 15B is situated at the distal surface of the toroidal balloon 15A. To accomplish the placement of the guide wire 12 into a specific pulmonary vein, it would be advantageous to make either or both the guide wire 12 and/or the guiding catheter 10 steerable and deflectable from outside the patient's body. Such devices are well known in the field of interventional cardiology.

As seen in FIG. 2, after the balloons 15A and 15B have been inflated, the distal portion 15 of the two-balloon catheter is pushed forward until the distal surface of the toroidal balloon 15A and the circumferential rf ablation wire 16 are pressed against the endocardial surface of the left atrium that is in close proximity to and surrounds the ostium of the pulmonary vein. The diameter formed by the rf ablation wire 16 after the toroidal balloon 15B is inflated is approximately 1-4 mm larger than the diameter of the pulmonary vein near its ostium. Thus, the diameter of the deployed ablation wire 16 should be between 5 and 12 mm depending on the diameter of the ostium of the pulmonary vein. The ablation of the tissue of the left atrium surrounding the ostium of pulmonary vein is then accomplished by electrical currents generated in the tissue of the left atrium by means of the rf ablation wire 16 and (typically) large electrode(s) placed on the patient's skin. Optimally, the large surface indifferent electrode on the patient's skin would be placed on the left side of his or her chest extending around from the back to the front of the chest. Alternatively to rf ablation, cryogenic or very hot fluid can be advanced through a tube that enters the toroidal balloon 15A so as to create an appropriate lesion in the tissue of the left atrium that surrounds the ostium of the pulmonary vein. Whatever the modality for creating this ablation of the tissue of the left atrium, the goal is to prevent undesired electrical conduction at or near the ostium of the pulmonary vein that is the cause of the patient's atrial fibrillation.

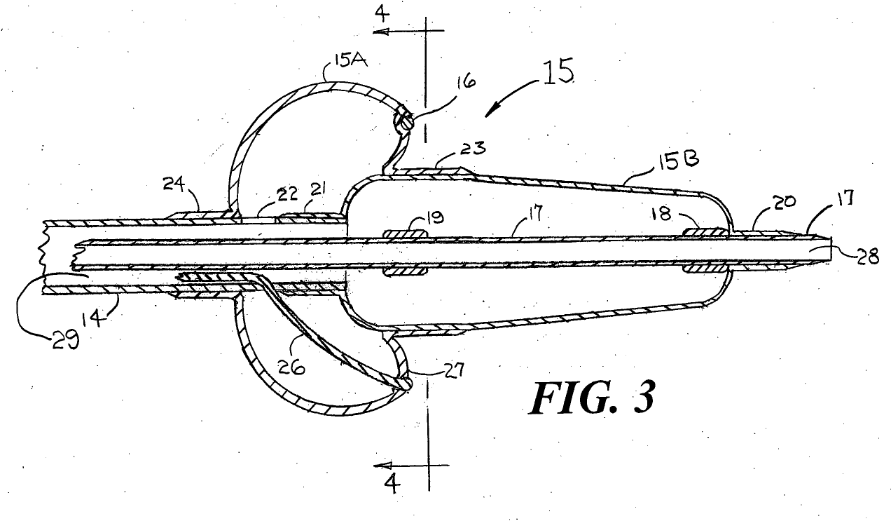

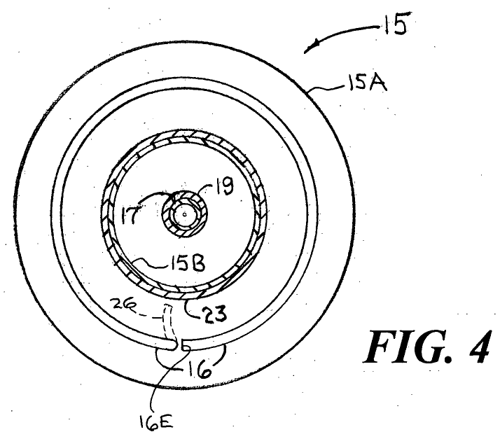

Also shown in FIG. 2 is the outer shaft 14 of the two-balloon catheter and the distal end of the inner shaft 17. Several of the features of the distal portion 15 of the two-balloon catheter are shown in greater detail in FIGS. 3 and 4.

If mapping of the patient's atrium shows that atrial fibrillation has been eliminated by the ablation of the left atrial tissue near a single pulmonary vein, then the entire apparatus to perform the ablation can be removed from the patient's body. If however, there is a need for performing this procedure at a total of two, three or even four of the pulmonary veins, then that can be successively accomplished. This is done by deflating the two balloons 15A and 15B of the two-balloon catheter, advancing the guide wire 12 to the next pulmonary vein, advancing the distal portion 15 of the two-balloon catheter over the guide wire 12 and into that next pulmonary vein and then inflating the tapered balloon 15B and the toroidal balloon 15A. It should be understood that the tapered balloon 15B could be inflated to a diameter just smaller than the diameter of the pulmonary vein near its ostium, the distal portion 15 would then be advanced until the ablation wire 16 was firmly pushed against the endocardial surface of the left atrium, and then the inflation of the tapered balloon 15B would be increased so that the tapered balloon 15B fits snugly into the pulmonary vein. This procedure would guarantee that the ablation wire 16 would be evenly placed at a uniform distance around the ostium of the pulmonary vein.

The method for ablating the tissue of the left atrium surrounding each of the pulmonary veins to be treated would then be accomplished until the doctor performing the procedure is convinced that all that can be done has been done to minimize the risk of recurrence of the patient's atrial fibrillation. Determination that the result has been successful can be accomplished by mapping of the electrical patterns in the heart or by other means such as an ECG and/or clinical follow-up.

FIG. 3 shows the distal portion 15 of the two-balloon catheter that can be used for ablation of the left atrial tissue that surrounds the ostium of one or more pulmonary vein or the superior vena cava. Shown in FIG. 3 is the inner shaft 17 having an interior lumen 28 through which the guide wire 12 (of FIGS. 1 and 2) is placed. Also shown in FIG. 3 is the outer shaft 14 having an interior annular lumen 29 through which fluid can be injected or removed to successively inflate and deflate the tapered balloon 15B and the toroidal balloon 15A. The distal end 20 of the tapered balloon 15B is sealed onto the inner shaft 17 and the proximal end 21 of the tapered balloon 15B is sealed onto the distal end of the outer shaft 14. The distal end 23 of the toroidal balloon 15A is attached to a proximal portion of the tapered balloon 15B and the proximal end 24 of the toroidal balloon 15A is sealed onto the outer shaft 14. Both the tapered balloon 15B and the toroidal balloon 15A are shown in FIG. 3 in their inflated state. The balloon material for each of the two balloons 15A and 15B should be somewhat compliant so that, after the balloons 15A and 15B have been inflated to their nominal diameter, a change in pressure of about one atmosphere should change the diameter of each balloon by about 0.05 mm to about 0.5 mm depending on the elastomer chosen and the wall thickness of each balloon. The pressure to deploy the balloons 15A and 15B to their nominal diameter should be between 2 and 6 atms. The toroidal balloon 15A may advantageously be made from silicone rubber so that it is truly elastic. The tapered balloon 15B should be made from a compliant elastomer as is well known in the field of interventional cardiology. Thus the distal end 23 of the toroidal balloon 15A which is located at the proximal portion of the tapered balloon 15B can be adjusted by means of fluid pressure to have an outer diameter that is just equal to or slightly smaller than the diameter of the pulmonary vein where the balloon 15B is placed. After inflation to that pressure is accomplished, the distal surface 27 of the toroidal balloon 15A is pushed forward until the circumferential rf ablation wire 16 is pushed firmly against the atrial tissue. The wire 16 could be made from a radiopaque metal such as tantalum to aid in its positioning. Also, the positioning of the wire 16 can be aided by the radiopaque proximal marker band 19 which is positioned to have its longitudinal center to be co-planar with the wire 16. The distal radiopaque marker band 18 helps the operator to position the two-balloon catheter during the procedure.

When the rf ablation wire 16 is pushed against the endocardial surface of the left atrium surrounding the ostium of the pulmonary vein, an rf ablation current is created into the tissue of the left atrium that surrounds the ostium of the pulmonary vein. The level and duration of the ablation current is adjusted to accomplish electrical isolation from the pulmonary vein without severely damaging the atrial or pulmonary vein tissue. To accomplish this, the operator could begin at a decreased current for a short time (1-1,000 milliamps for 0.1-100 seconds). The current can be increased in amplitude or duration until mapping or ECG shows that atrial fibrillation has been successfully treated. An important method to optimize the treatment level is to use endocardial echo to note when gas bubbles appear around the rf ablation active electrode 16. As soon as gas bubbles appear, the rf current is immediately stopped. It is likely that experience will dictate that certain amplitude and current (or other ablative means) is safe and effective in a large majority of cases. If so, then this amount of energy, or this approach could be used qualitatively or empirically without the need for observation of bubbles or the immediate cessation of atrial fibrillation. For the ablation, the circumferential wire 16 serves as the active electrode and a large surface electrode placed on the patient's skin acts as the indifferent electrode. The optimal position of the indifferent electrode may be to have it wrapped around the left side of the patient's chest in the vicinity of the heart.

Inflation of the balloons 15A and 15B can be accomplished by pressurized fluid (typically radiopaque contrast diluted with normal saline) that is injected through the annular lumen 29. Before any liquid is used to pressurize the balloons 15A and 15B, they should be subjected to a vacuum and then inflated with carbon dioxide gas and then have vacuum applied again. In this way, there can be no air bubbles that could be left in the two-balloon catheter that could come out of a burst balloon that could cause a stroke to occur. The inflation fluid would fill the toroidal balloon 15A by passing through at least one hole 22 located near the distal end of the outer shaft 14. Instead of a wire 26, a tube (not shown) whose open distal end lies in the toroidal balloon 15A could be used to flow either very hot or very cold (cryogenic) fluid thru the toroidal balloon 15A to create the appropriate lesion in the atrial tissue. Because the flow would be only through the toroidal balloon 15A, the tapered balloon 15B would not get as hot or as cold as the surface of the toroidal balloon 15A that is in intimate contact with the atrial tissue surrounding the ostium of the pulmonary vein. Thus damage to the tissue of the pulmonary vein could be avoided or reduced while damage to the tissue of the left atrium surrounding the ostium of the pulmonary vein would be much more severe as is desired. Alternatively, the wire 16 could be a small diameter plastic tube (not shown) that has both its entry port and exit port lying outside the patient's body beyond the proximal end of the two-balloon catheter with an entry tube and exit tube (each like the wire 26) that each lies in the annular lumen 29. Such a tube could be circumferentially placed like the rf ablation wire 16 so that it could be pushed against the tissue of the left atrium that surrounds the ostium of the pulmonary vein. In that way, heating or cooling for ablation could be accomplished through such a tubular system without the blood (in left atrium) or pulmonary vein tissue being exposed to inordinately hot or cold temperatures. This could avoid the creation of blood clots or frozen crystals in the blood of the left atrium and avoid damage to the tissue of the pulmonary vein.

FIG. 3 also shows a return electrical wire 26 that is conductively joined to the circumferential rf ablation wire 16. This wire 26 would pass through the annular lumen 29 that is situated between the outer surface of the inner shaft 17 and the inner surface of the outer shaft 14. Because the distal surface 27 of the toroidal balloon 15A is pushed tight against the wall of the left atrium, most of the blood is excluded so that the rf ablation should not produce any significant level of thrombus of the blood in the heart. This is very important to prevent any blood clot from embolizing to a cerebral artery where it could cause a stroke. Cryogenic fluid in the toroidal balloon 15A could cause ice crystals to form in the blood of the left atrium. That is a much less important problem because such crystals would melt in the cerebral circulation before they can cause any permanent damage by the ischemia that they could temporarily create.

Although the tapered balloon 15B is shown to have a tapered shape, it could also be in the generally cylindrical shape of most angioplasty balloons. Furthermore, the balloons 15A and 15B could be a single balloon that has about the same outside shape as the two balloons 15A and 15B. Thus, the two-balloon catheter system described herein also has the meaning of being a single balloon catheter system that can accomplish the same treatment for atrial fibrillation.

FIG. 4 shows the cross section of the distal portion 15 of the two-balloon catheter at section 4-4 of FIG. 3. FIG. 4 shows the outline of the toroidal balloon 15A, the cross section at a proximal portion of the tapered balloon 15B, the rf ablation wire 16, the inner shaft 17, the proximal radiopaque marker band 19 and the cross section of the distal end 23 of the balloon 15A. Of interest in FIG. 4 near the bottom of the drawing is the distal end 16E of the rf ablation wire 16 and the end of wire 16 that is connected to the wire 26 that lies inside the toroidal balloon 15A, then enters the annular lumen 29 and finally exits through the proximal end of the two-balloon catheter outside the patient's body. The wire 16 is the active electrode for rf ablation and a large surface electrode on the patient's skin is the indifferent electrode. Through the active and indifferent electrodes, a high intensity, localized electric current is created in the tissue of the left atrium that creates the lesion that stops the patient from having atrial fibrillation.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A two-balloon catheter system for the treatment of atrial fibrillation, the system including:
an inner shaft designed to be advanced over a guide wire;
an outer shaft placed co-axially around the inner shaft and having a length that extends for most of the length of the inner shaft;
a tapered balloon having a distal end fixedly attached at or near the distal end of the inner shaft and a proximal end fixedly attached to the distal end of the outer shaft, the tapered balloon being formed from a compliant elastomer so that after the tapered balloon is expanded to its nominal diameter, an increase in one atmosphere of pressure can further increase the diameter of the tapered balloon by at least 0.05 mm;
a toroidal balloon having a distal end that is fixedly attached to a proximal portion of the tapered balloon and a proximal end that is fixedly attached near the distal end of the outer shaft, the toroidal balloon having a distal surface that is designed to be placed firmly against the intracardiac surface of the left atrium when the tapered balloon is placed through the ostium of a pulmonary vein and then inflated, the toroidal balloon including means for ablating the tissue of the left atrium that is in close proximity to the ostium of the pulmonary vein when the distal surface of the toroidal balloon is pressed against the intracardiac surface of the left atrium that surrounds the ostium of that pulmonary vein.

2. The system of claim 1 where the tapered balloon has a smaller diameter near its distal end as compared to the diameter at its proximal portion.

3. The system of claim 1 where the toroidal balloon is inflated and deflated by means of at least one hole in the outer shaft that is situated between the proximal ends of the tapered balloon and the toroidal balloon.

4. The system of claim 1 where the distal surface of the toroidal balloon includes a means for ablating the tissue of the left atrium that surrounds the ostium of the pulmonary vein.

5. The system of claim 4 where the means for ablating is heating by electrical means of the tissue of the left atrium.

6. The system of claim 5 where the electrical means is by rf heating.

7. The system of claim 4 where the ablation is accomplished by a cryogenic fluid.

8. The system of claim 4 where the ablation is accomplished using a heated fluid.

9. The system of claim 4 where the ablation is carried out by a generally circumferential structure located on the distal surface of the toroidal balloon, the circumferential structure generally surrounding the ostium of the pulmonary vein.

10. The system of claim 9 where the diameter of the circumferential structure is approximately 1-4 mm greater in diameter as compared to the diameter of the pulmonary vein near its ostium.

11. The system of claim 9 where the circumferential structure is an electrically conducting wire.

12. The system of claim 9 where the circumferential structure is a small diameter hollow tube through which either a heated or a cooled fluid can flow.

13. The system of claim 1 where the two-balloon catheter is advanced through the interatrial septum within a guiding catheter that was previously placed through the interatrial septum.

14. The system of claim 1 where the guide wire used for the treatment of atrial fibrillation is between 0.035 and 0.038 inches in diameter.

15. The system of claim 1 where the toroidal balloon is made from a highly elastic elastomer such as silicone rubber.

16. A method for the treatment of atrial fibrillation, the method including the following steps:
a) an introducer sheath is placed into the left or right femoral vein,
b) a guide wire is advanced through the femoral vein, through the inferior vena cava and then into the right atrium,
c) using standard trans-septal puncture technique, a guide wire is placed across the interatrial septum from the right into the left atrium,
d) further dilatation of the interatrial septum puncture site is able to be performed with a balloon angioplasty catheter, if necessary,
e) appropriate anticoagulation is given to minimize the risk of left atrial thrombus during the rest of the manipulations,
f) a special purpose guiding catheter including a dilator is advanced over the guide wire and through the inferior vena cava and into the right atrium,
g) the dilator and guiding catheter are then pushed through the interatrial septum from the right atrium into the left atrium, using the guide wire already placed in the left atrium,
h) the dilator is removed,
i) a specially designed two-balloon catheter is then advanced over the guide wire and through the guiding catheter until its distal end is situated in the left atrium,
j) the guide wire and/or the guiding catheter are manipulated until the guide wire is placed into the first targeted pulmonary vein,
k) the guidance of the guide wire and the balloon ablation catheter into each of the targeted pulmonary veins (up to four) is guided by intra-cardiac or trans-esophageal ultrasound imaging, or other real-time imaging modalities,
l) the tapered balloon and the toroidal balloon are then advanced until the proximal portion of the tapered balloon is situated just proximal to the ostium of that pulmonary vein,
m) the tapered balloon and the toroidal balloon are then inflated to a pressure such that the proximal portion of the tapered balloon just fits within the ostial region of the pulmonary vein,
n) the balloons are then pushed forward until the distal surface of the toroidal balloon is in contact with, and applies some pressure onto, the endocardial surface of the left atrium surrounding the ostium of the pulmonary vein,
o) ablation of the tissue of the left atrium surrounding the ostium of the pulmonary vein is then applied by any well known method such as the use of rf energy, cryogenic cooling or heating with a high temperature fluid or heated electrical wire,
p) the application of the ablative means is then discontinued and the balloons are deflated,
q) if it is desired to ablate the region of the left atrium near the ostium of a second, third or fourth pulmonary vein, then the steps 10-16 above are repeated for each region where it is desired to perform tissue ablation of the left atrium, and
r) when treatment of the last region of the left atrium that is to be ablated is completed, all the apparatus used (viz., the guide wire, two-balloon catheter, guiding catheter and introducer sheath, etc.) are then removed from the patient's body.

17. The method of claim 16 further including the step of ablating the endocardial tissue of the right atrium that is in close proximity to the ostium of the superior vena cava.

18. The method of claim 16 further including the step of utilizing the appearance of gas bubbles as an indication that the rf ablation electric current should be turned off.

19. The method of claim 16 further including the step of wrapping an indifferent electrode onto the patient's skin at a location that is generally around the patient's chest on his or her left side in the region of the patient's heart.
